(19) United States
(12) Patent Application Publication
(10) Pub. No.: US 2014/0296290 A1
(43) Pub. Date: Oct. 2, 2014

(54) THERAPEUTIC USES OF COMPOUNDS HAVING AFFINITY TO THE SEROTONIN TRANSPORTER, SEROTONIN RECEPTORS AND NORADRENALIN TRANSPORTER

(71) Applicants: H. Lundbeck A/S, Valby-Coppenhagen (DK); Takeda Pharmaceuticals North America, Inc., Deerfield, IL (US)

(72) Inventors: Silke Miller, Newbury Park, CA (US); Nanco Hefting, Virum (DK); Eva Elstrup Jensen, Jyderup (DK); Aneil Batra, Chicago, IL (US); Jin Chon, Barrington, IL (US)

(21) Appl. No.: 14/279,504

(22) Filed: May 16, 2014

Related U.S. Application Data
(63) Continuation of application No. 12/747,403, filed on Nov. 11, 2010, now abandoned.
(60) Provisional application No. 61/013,884, filed on Dec. 14, 2007.

(30) Foreign Application Priority Data
Dec. 14, 2007 (DK) PA200701791
Dec. 17, 2007 (DK) PA200701798

(51) Int. Cl. A61K 31/451

(52) U.S. Cl. CPC A61K 31/451
USPC 514/317

(57) ABSTRACT

Therapeutic uses of 4-[2-(4-methylphenylsulfanyl)phenyl]piperidine and therapeutically acceptable salts thereof are provided.

### FIELD OF THE INVENTION

The present invention relates to therapeutic uses of compounds having serotonin and noradrenalin transport inhibiting activity in combination with affinity for serotonin receptors.

### BACKGROUND OF THE INVENTION

Selective serotonin reuptake inhibitors (SSRI) have for years been favoured by physicians for the treatment of many CNS diseases, such as depression and anxiety because they are effective and have a safety profile which is favourable compared to the previous generation of CNS drugs, i.e. the so-called tri-cyclics. Nevertheless, SSRI's are hampered by a significant fraction of non-responders, i.e. patients who do not or who do not fully respond to the treatment. Moreover, typically an SSRI does not begin to show an effect until after weeks of treatment. Finally, although SSRI's typically give rise to less adverse effects than tri-cyclics, the administration of SSRI's often brings about adverse effects, such as e.g. sleep disruption.

It is known that a combination of inhibition of the serotonin transporter (SERT) and an activity on one or more serotonin receptors may result in a faster increase in the serotonin level. It has been reported that the combination of pindolol, which is a 5-HT_{1A} partial agonist, with a serotonin reuptake inhibitor gives rise to faster onset of effect [Psych. Res., 125, 81-86, 2004]. Similarly, it has been found that the combination of a serotonin reuptake inhibitor with a compound having 5-HT_{2C} antagonistic or inverse agonistic effect (compounds having a negative efficacy at the 5-HT_{2C} receptor) provides a considerable increase in the level of 5-HT in terminal areas, as measured in microdialysis experiments [WO 01/41701]. As the therapeutic effect of SERT compounds is believed to be caused by the increase in the serotonin level induced, a combination of these activities would imply a shorter onset of the therapeutic effect in the clinic and an augmentation or potentiation of the therapeutic effect of the serotonin reuptake inhibitor.

Several neurotransmitters are presumed to be involved in the neuronal events regulating cognition. In particular, the cholinergic system plays a prominent role in cognition, and compounds affecting the cholinergic system are thus potentially useful for the treatment of cognitive impairment. Compounds affecting the 5-HT₃ receptor are known to affect the cholinergic system, and they may as such be useful in the treatment of cognitive impairment.

It is well-known that antagonists of the 5-HT₂ receptor and in particular 5-HT_{2A} and 5-HT_{2C} antagonists may be useful in the treatment of sleep disorders [Neuropharmacol., 33, 467-471, 1994; Bioorg. Med. Chem. Lett., 15, 3665-3669, 2005].

Thus compounds that are SERT inhibitors and inhibitors of the 5-HT_{2A/C} and 5-HT₃ receptor would be expected to be particularly useful in the treatment of cognitive impairment in patients also suffering from a disease that would benefit from an increase in the serotonin level. Such therapeutic intervention would be expected to give rise to fewer of the adverse effects e.g. on sleep often associated with the use of SERT compounds. These adverse effects include problems with sleep initiation and maintenance, problems with insomnia, suppressed REM sleep, increased sleep latency, less efficient sleep, increase in nocturnal awakenings, and fragmentation of sleep [Hum. Psychopharm. Clin. Exp., 20, 533-559, 2005; Int. Clin. Psychpharm., 21 (suppl 1), S25-S29, 2006].

The international patent application published as WO 2003/029232 discloses e.g. the compound 4-[2-(4-methylphenylsulfanyl)phenyl]piperidine as a free base and the corresponding HCl salt. The compound is reported to be an inhibitor of the serotonin transporter and the 5-HT_{2C} receptor, and is said to be useful for the treatment of affective disorders, e.g. depression and anxiety.

WO 2007/144006 discloses further pharmaceutical uses of 4-[2-(4-methylphenyl-sulfanyl)phenyl]piperidine and also that the compound in addition to being a serotonin transport inhibitor is a noradrenaline transport inhibitor and an antagonist of the 5-HT_{2A} and 5-HT₃ receptor and the α₁ adrenergic receptor.

### SUMMARY OF THE INVENTION

The present inventors have found that in addition to the already known pharmacological profile, 4-[2-(4-methylphenylsulfanyl)-phenyl]piperidine is a potent inhibitor of the noradrenalin reuptake, and an antagonist of the serotonin receptor 3 (5-HT₃). Accordingly, the invention relates to methods of treating certain diseases comprising the administration of 4-[2-(4-methylphenylsulfanyl)-phenyl]piperidine and pharmaceutically acceptable salts thereof to a patient in need thereof.

In one embodiment, the invention relates the use of 4-[2-(4-methylphenyl-sulfanyl)phenyl]piperidine and pharmaceutically acceptable salts thereof in the manufacture of medicaments for the treatment of certain diseases.

In one embodiment, the invention relates to 4-[2-(4-methylphenylsulfanyl)-phenyl]piperidine and pharmaceutically acceptable salts thereof for use in the treatment of certain diseases.

### FIGURES

FIG. 1: X-ray diffraction pattern of the HBr addition salt of compound I

FIG. 2: X-ray diffraction pattern of the HBr addition salt, solvate of compound I

FIG. 3: X-ray diffraction pattern of the DL-lactic acid addition salt of compound I

FIG. 4: X-ray diffraction pattern of the L-aspartic acid addition salt (1:1) of compound I in mixture with L-aspartic acid

FIG. 5: X-ray diffraction pattern of the L-aspartic acid addition salt hydrate (1:1) of compound I in mixture with L-aspartic acid

FIG. 6: X-ray diffraction pattern of the glutamic acid addition salt (1:1) of compound I in mixture with glutamic acid monohydrate

FIG. 7: X-ray diffraction pattern of the glutaric acid addition salt (1:1) of compound I

FIG. 8: X-ray diffraction pattern of the malonic acid addition salt (1:1) of compound I, α-form

FIG. 9: X-ray diffraction pattern of the malonic acid addition salt of compound I, β-form

FIGS. 10A and 10B: Acetylcholine levels in the prefrontal cortex and ventral hippocampus upon administration of compound I

FIG. 11: Dopamine levels in prefrontal cortex upon administration of compounds of the present invention

### DETAILED DESCRIPTION OF THE INVENTION

The present invention relates to the use of compound I, which is 4-[2-(4-methylphenylsulfanyl)-phenyl]piperidine and pharmaceutically acceptable salts thereof. The structure of 4-[2-(4-methylphenylsulfanyl)-phenyl]piperidine is

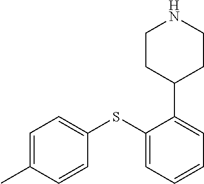 [SMILES: Cc1ccc(Sc2ccccc2C2CCNCC2)cc1]

In one embodiment, said pharmaceutically acceptable salts are acid addition salts of acids that are non-toxic. Said salts include salts made from organic acids, such as maleic, fumaric, benzoic, ascorbic, succinic, oxalic, bis-methylenesalicylic, methanesulfonic, ethanedisulfonic, acetic, propionic, tartaric, salicylic, citric, gluconic, lactic, malic, malonic, mandelic, cinnamic, citraconic, aspartic, stearic, palmitic, itaconic, glycolic, p-aminobenzoic, glutamic, benzenesulfonic, theophylline acetic acids, as well as the 8-halotheophyllines, for example 8-bromotheophylline. Said salts may also be made from inorganic salts, such as hydrobromic, hydrochloric, sulfuric, sulfamic, phosphoric and nitric acids. Additional useful salts are listed in the table in example 3 (table 1).

In one embodiment, the invention provides the use of compound I provided compound I is not the free base in a non-crystalline form or the hydrochloric acid salt in a crystalline form.

Oral dosage forms, and in particular tablets and capsules, are often preferred by the patients and the medical practitioner due to the ease of administration and the consequently better compliance. For tablets and capsules, it is preferable that the active ingredients are crystalline. In one embodiment, compound I is crystalline, and in particular provided it is not the hydrochloric acid salt.

Crystals used in the present invention may exist as solvates, i.e. crystals wherein solvent molecules form part of the crystal structure. The solvate may be formed from water, in which case the solvates are often referred to as hydrates. Alternatively, the solvates may be formed from other solvents, such as e.g. ethanol, acetone or ethyl acetate. The exact amount of solvate often depends on the conditions. For instance, hydrates will typically loose water as the temperature is increased or as the relative humidity is decreased. Compounds, which do not change or which change only little when conditions, such as e g humidity, change are generally regarded as better suited for pharmaceutical formulations. It is noted that the HBr addition salt does not form hydrates when precipitated from water whereas compounds such as the succinate, malate and tartrate acid addition salts do.

Some compounds are hygroscopic, i.e. they absorb water when exposed to humidity. Hygroscopicity is generally regarded as an undesired property for compounds, which are to be presented in a pharmaceutical formulation, in particular in a dry formulation, such as tablets or capsules. In one embodiment, the invention provides crystals with low hygroscopicity.

For oral dosage forms using crystalline active ingredients it is also beneficial if said crystals are well-defined. In the present context, the term “well-defined” in particular means that the stoichiometry is well-defined, i.e. that the ratio between the ions forming the salt is the ratio between small integers, such as 1:1, 1:2, 2:1, 1:1:1, etc. In one embodiment, the compounds of the present invention are well-defined crystals.

The solubility of an active ingredient is also of significance for the choice of dosage form as it may have a direct impact on bio-availability. For oral dosage forms, a higher solubility of the active ingredient is generally believed to be beneficial as it increases the bio-availability. Some patients, e.g. elderly patients may have difficulties swallowing tablets, and oral drop solutions may be a suitable alternative avoiding the need for swallowing tablets. In order to limit the volume of an oral drop solution, it is necessary to have a high concentration of the active ingredient in the solution, which again requires a high solubility of the compound. As shown in table 3, DL-lactic acid, L-aspartic acid, glutamic acid, glutaric acid and malonic acid addition salts have exceptionally high solubility.

Crystal forms impact the filtration and processing properties of a compound. Needle formed crystals tend to be more difficult to handle in a production environment as filtration becomes more difficult and time consuming The exact crystal form of a given salt may depend e.g. on the conditions under which the salt was precipitated. The HBr acid addition salt used in the present invention grows needle-shaped, solvated crystals when precipitated from ethanol, acetic acid and propanol, but crystals of a non-hydrated form, which are not needle-shaped, when HBr addition salt is precipitated from water, providing superior filtration properties.

Table 3 also depicts the Resulting pH, i.e. the pH in the saturated solution of the salt. This property is of importance because moisture can never be completely avoided during storage and the accumulation of moisture will give rise to a pH decrease in or on a tablet comprising a low Resulting pH salt, which may decrease shell life. Moreover, a salt with a low resulting pH may give rise to corrosion of process equipment if tablets are made by wet granulation. The data in table 3 suggests that the HBr, HCl and adipic acid addition salts may be superior in this respect.

In one embodiment, the compound used in the present invention, i.e. the compound of formula I, is the HBr addition salt

In one embodiment, the compound used in the present invention is the DL-lactic acid addition salt, and in particular the 1:1 salt.

In one embodiment, the compound used in the present invention is the L-aspartic acid addition salt, and in particular the 1:1 salt.

In one embodiment, the compound used in the present invention is the glutamic acid addition salt, and in particular the 1:1 salt.

In one embodiment, the compound used in the present invention is the glutaric acid addition salt, and in particular the 1:1 salt.

In one embodiment, the compound used in the present invention is the malonic acid addition salt, and in particular the 1:1 salt that is found to exist in two polymorphic modifications α and β of which the β form is believed to be the most stable based on a lower solubility.

In one embodiment, the compound used in the present invention is in a purified form. The term “purified form” is intended to indicate that the compound is essentially free of other compounds or other forms, i.e. polymorphs of said compound, as the case may be.

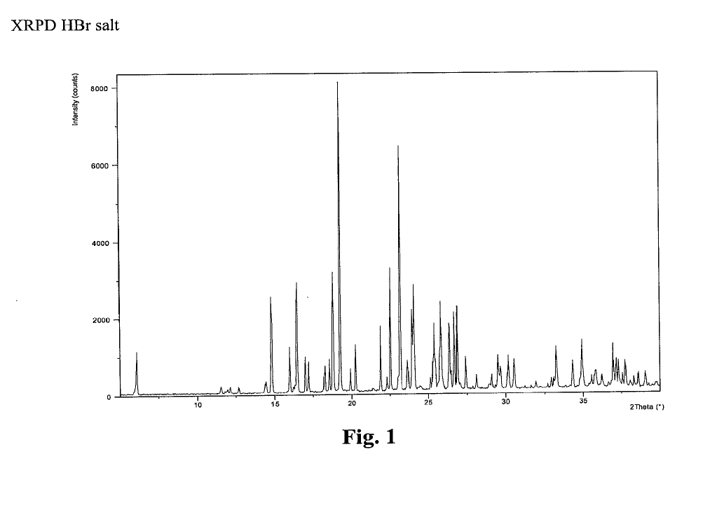

In one embodiment, the compound used the present invention is the HBr addition salt in a crystalline form, in particular in a purified form. In a further embodiment, said HBr salt has peaks in an X-ray powder diffractogram (XRPD) at approximately 6.08°, 14.81°, 19.26° and 25.38°2θ, and in particular said HBr salt has an XRPD as depicted in FIG. 1.

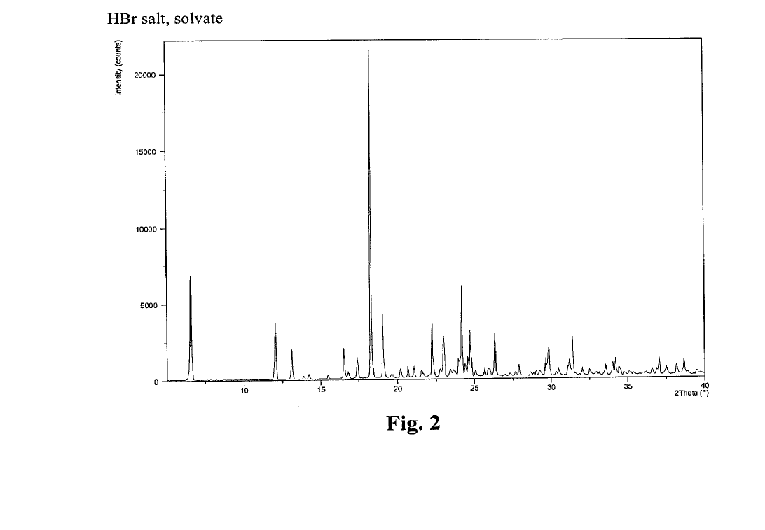

In one embodiment, the compound used in the present invention is the DL-lactic acid addition salt (1:1) in a crystalline form, in particular in a purified form. In a further embodiment, said DL-lactic acid addition salt has peaks in a XRPD at approximately 5.30°, 8.81°, 9.44° and 17.24°2θ, and in particular said DL lactic acid addition salt has an XRPD as depicted in FIG. 2

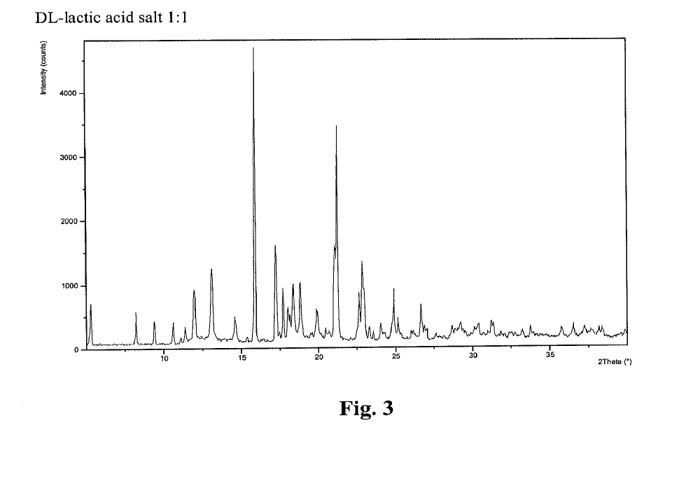

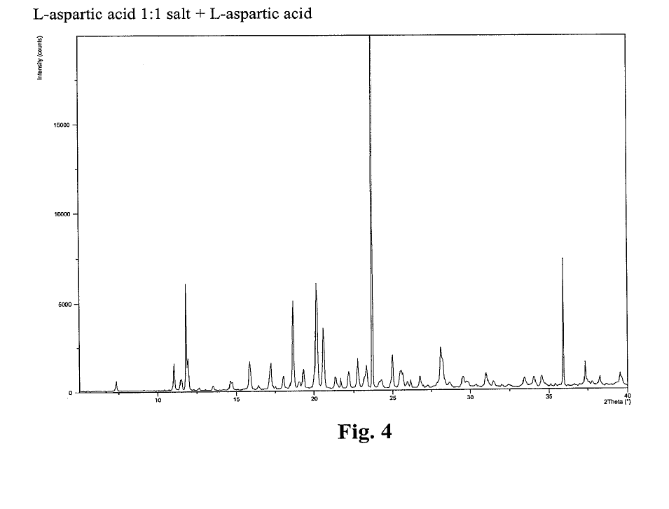

In one embodiment, the compound used in the present invention is the L-aspartic acid addition salt (1:1) in a crystalline form, in particular in a purified form. In a further embodiment, said L-aspartic acid addition salt is unsolvated and has peaks in a XRPD at approximately 11.05°, 20.16°, 20.60° and 25.00°2θ. In one embodiment, said L-aspartic salt, when mixed with L-aspartic acid, has an XRPD as depicted in FIG. 3. In one embodiment, said L-aspartic acid addition salt is a hydrate, in particular in a purified form. In a further embodiment, said L-aspartic acid addition salt hydrate has peaks in a XRPD at approximately 7.80°, 13.80°, 14.10° and 19.63°2θ. In one embodiment, said L-aspartic addition salt hydrate, when mixed with L-aspartic acid, has an XRPD as depicted in FIG. 4.

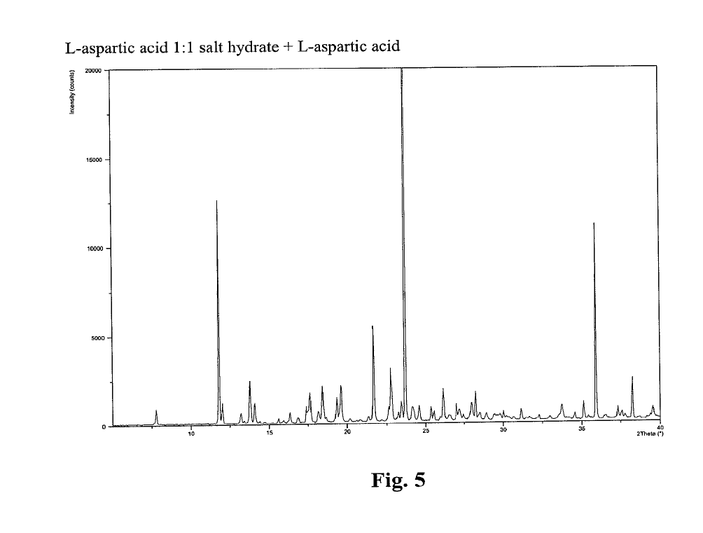

In one embodiment, the compound used in the present invention is the glutamic acid addition salt (1:1) in a crystalline form, in particular in a purified form. In a further embodiment, said glutamic acid addition salt has peaks in a XRPD at approximately 7.71°, 14.01°, 19.26° and 22.57°2θ, and in particular said glutamic acid salt, when mixed with glutamic acid monohydrate, has an XRPD as depicted in FIG. 5.

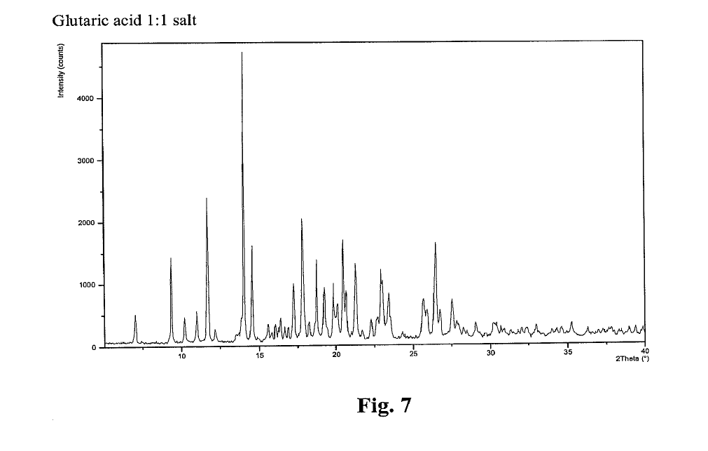

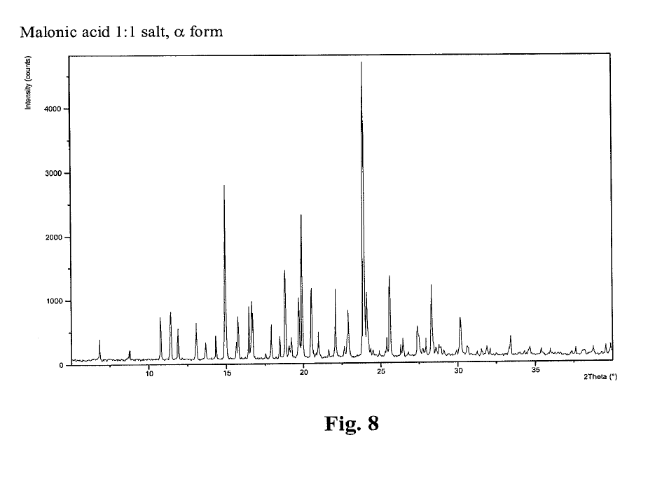

In one embodiment, the compound used in the present invention is the malonic acid addition salt (1:1) in a crystalline form, in particular in a purified form. In a further embodiment, said malonic acid addition salt is the α-form and has peaks in a XRPD at approximately 10.77°, 16.70°, 19.93° and 24.01°2θ, or said malonic acid addition salt is the β-form and has peaks in a XRPD at approximately 6.08°, 10.11°, 18.25° and 20.26°2θ and in particular said malonic acid addition salt has an XRPD as depicted in FIG. 7 or 8.

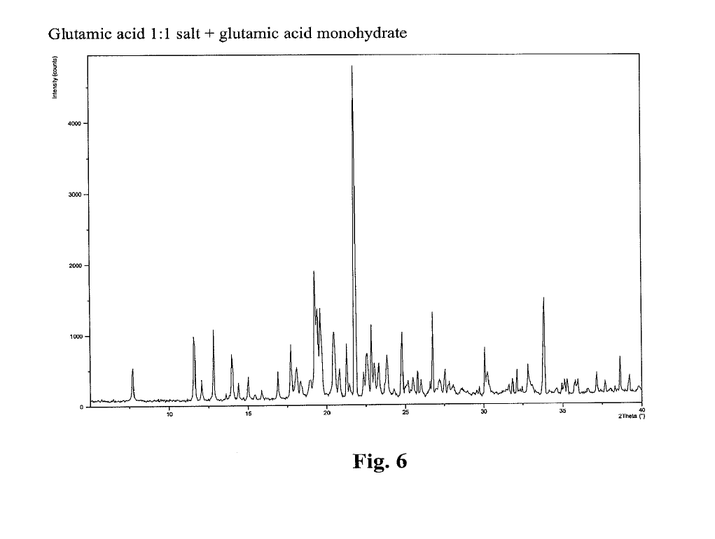

In one embodiment, the compound used in the present invention is the glutaric acid addition salt (1:1) in a crystalline form, in particular in a purified form. In a further embodiment, said glutaric acid addition salt has peaks in a XRPD at approximately 9.39°, 11.70°, 14.05° and 14.58°2θ, and in particular said glutaric acid addition salt has an XRPD as depicted in FIG. 6.

The pharmacological profile of compound I encompasses serotonin and noradrenalin reuptake inhibition and 5-HT₃ antagonism. These activities suggest that compound I may be particularly useful in the treatment of pain, e.g. chronic pain [Clin. Ther. 26, 951-979, 2004; Exp. Opin. Ther. Targets, 11, 527-540, 2007]. In fact, the data provided in example 6 shows that compound I is useful in the treatment of pain. Compound I may be used in the treatment of pain, or in the treatment of pain associated with other diseases, e.g. CNS diseases and in particular depression or anxiety.

Data presented in example 4 shows that compound I effects an increase in the extra cellular level of acetylcholine in the prefrontal cortex and the hippocampus. There is longstanding clinical evidence that increasing acetylcholine levels in the brain is instrumental in treating Alzheimer's disease and cognitive impairment in general, cf. the use of acetylcholine esterase inhibitors in the treatment of Alzheimer's disease.

Data presented in example 5 shows that compound I effects an increase in the extra cellular level of dopamine in the prefrontal cortex. Based on the improvement in the executive and cognitive functions in Parkinson's patients upon treatment with dopamine receptor agonists or dopa, it has been suggested that the dopamine level also plays a significant role for cognition. Cognitive impairment is common in geriatric depression or anxiety, i.e. depression or anxiety in the elderly population. The data shown for the compounds used in the present invention suggest that these compounds are useful in the treatment of depression or anxiety in the elderly population.

Cognitive deficits or cognitive impairment include a decline in cognitive functions or cognitive domains, e.g. working memory, attention and vigilance, verbal learning and memory, visual learning and memory, reasoning and problem solving e.g. executive function, speed of processing and/or social cognition. In particular, cognitive deficits or cognitive impairment may indicate deficits in attention, disorganized thinking, slow thinking, difficulty in understanding, poor concentration, impairment of problem solving, poor memory, difficulties in expressing thoughts and/or difficulties in integrating thoughts, feelings and behaviour, or difficulties in extinction of irrelevant thoughts. The terms “cognitive deficits” and “cognitive impairment” are intended to indicate the same and are used interchangeably.

In one embodiment, compound I may also be used for the treatment of patients who in addition to a cognitive impairment are also diagnosed with another CNS disorder, such as affective disorders, such as depression; generalised depression; major depressive disorder; anxiety disorders including general anxiety disorder and panic disorder; obsessive compulsive disorder; schizophrenia; Parkinson's; dementia; AIDS dementia; ADHD; age associated memory impairment; Down's syndrome, tryptophane hydrolase gene mutations, or Alzheimer's disease.

Cognitive impairment is among the classic features of depression, such as e.g. major depressive disorder. Cognitive disorders may to some extend be secondary to depression in the sense that an improvement in the depressive state will also lead to an improvement of the cognitive impairment. However, there is also clear evidence that cognitive disorders are, indeed, independent from depression. For instance, studies have shown persistent cognitive impairment upon recovery from depression [J. Nervous Mental Disease, 185, 748-754, 197]. Moreover, the differential effect of antidepressants on depression and cognitive impairments lends further support to the notion that depression and cognitive impairment are independent, albeit often co-morbid conditions. While serotonin and noradrenalin medicaments provide comparable improvements in depressive symptoms, several studies have shown that modulation of the noradrenergic system does not improve the cognitive functions as much as serotonin modulation [Brain Res. Bull., 58, 345-350, 2002; Hum Psychpharmacol., 8, 41-47, 1993].

The cognitive effects of compound I also make it useful in the treatment of psychomotor retardation. Psychomotor retardation is characterised by cognitive impairment and reduction of physical movements in a subject. Psychomotor retardation is often seen in depressed patients where it is indicative of severe depression. Patients suffering from psychomotor retardation have difficulties in handling daily activities, such as dressing, self-grooming and cooking, and in doing things that require movement, such as shopping.

Compound I has been used in a multiple dose clinical trial wherein 70 healthy volunteers were administered compound I at up to 30 mg/day or placebo. 49 subjects received active compound and 21 subjects received placebo. Vital signs including blood pressure were measured during the trial, and only at the highest dose were there signs of elevated blood pressure. This would seem to suggest that compound I does not give rise to increases in blood pressure at expected clinical doses, and consequently that compound I may be used in the treatment of patients with hypertension or patients with increased risk of hypertension.

The broad pharmacological profile of the compounds used in the present invention suggest that they are also useful in the treatment of depression in patients who do not or who do not adequately respond to treatment with SSRI.

The unique pharmacological profile of compound I makes the compound useful in the treatment of diseases selected from depression, such as severe depression, psychomotor retardation, dysthymic disorder, cyclothymia, mood disorder due to a generalised medical condition, substance induced depression, recurrent depression, single episode depression, paediatric depression, atypical depression, post-stroke depression, exhaustion depression, depression associated with gastrointestinal pain, such as IBS (irritable bowl syndrome), abuse, hostility, irritability, fatigue, anxiety (anxious depression), Lewy Body disease, Huntington's disease, or multiple sclerosis, general anxiety disorder associated with pain, seasonal affective disorder (SAD), depression or anxiety in patients with increased risk of hypertension, depression or anxiety in patients with sleep problems, stress related disorder, acute stress, dementia, mild cognitive impairment (MCI), cognitive impairment in schizophrenia or Parkinson's disease, age associated cognitive impairment, vascular dementia, leucariosis, small vessel disease, cognitive impairment associated with affective disorders, such as depression, generalised depression, major depressive disorder, anxiety disorders including general anxiety disorder and panic disorder, obsessive compulsive disorder, schizophrenia, Parkinson's disease, dementia, AIDS dementia, ADHD, age associated memory impairment, Down's syndrome, tryptophane hydrolase gene mutations, epilepsy, traumatic brain injury, or Asperger's syndrome, pre-, peri- or post-menstrual dysphoric disorder, pathological crying, autism, obesity, anorexia, bulimia and binge eating, impulse control disorder, intermittent explosive disorder, kleptomania, pyromania, pathological gambling, trichotillomania, conduct disorder, burn-out, stress, chronic fatigue syndrome, circadian rhythm disorder, sleep disorder; sleep-disordered breathing; hypopnea syndrome; behavioural disturbances, behavioural disturbances in the elderly, behavioural disturbances associated with dementia, compulsive and attention spectrum disorder associated with ADHD, Asperger's syndrome and autism, aggression and agitation in dementia and Alzheimer's disease, insulin resistance associated with HPA-axis hyperactivity, whiplash, fear of flying, elevators or small rooms, and amblyopia.

In this context, severe depression is intended to indicate depression wherein the patient scores above 30, such as above 32 or above 35 on the MADRS scale.

Thus in one embodiment, the invention provides a method for the treatment of a disease selected from psychomotor retardation; severe depression; dysthymic disorder; cyclothymia; mood disorder due to a generalised medical condition; substance induced depression; recurrent depression; single episode depression; paediatric depression; atypical depression; post-stroke depression; exhaustion depression; depression associated with gastrointestinal pain, IBS, abuse, hostility, irritability, fatigue, anxiety (anxious depression), Lewy Body disease, Huntington's disease, or multiple sclerosis; general anxiety disorder associated with pain; seasonal affective disorder (SAD); depression or anxiety in patients with increased risk of hypertension; depression or anxiety in patients with sleep problems; stress related disorder; acute stress; dementia; mild cognitive impairment (MCI); vascular dementia; leucariosis; small vessel disease; cognitive impairment associated with affective disorders, depression, generalised depression, major depressive disorder, anxiety disorders, general anxiety disorder, panic disorder, obsessive compulsive disorder, schizophrenia, Parkinson's disease, dementia, AIDS dementia, ADHD, age associated memory impairment, Down's syndrome, epilepsy, traumatic brain injury, Asperger's syndrome, and tryptophane hydrolase gene mutations; pre-, peri-, or post menopausal dysphoric disorder; pathological crying; autism; obesity; anorexia; bulimia; binge eating; impulse control disorder; intermittent explosive disorder; kleptomania; pyromania; pathological gambling; trichotillomania; conduct disorder; burn-out; stress; chronic fatigue syndrome; circadian rhythm disorder; sleep disorder; sleep-disordered breathing; hypopnea syndrome; behavioural disturbances; behavioural disturbances in the elderly; behavioural disturbances associated with dementia; compulsive and attention spectrum disorder associated with ADHD, Asperger's syndrome and autism; aggression and agitation in dementia and Alzheimer's disease; insulin resistance associated with HPA-axis hyperactivity; whiplash; fear of flying, elevators or small rooms; and amblyopia, the method comprising the administration of a therapeutically effective amount of compound I to a patient in need thereof.

In one embodiment, the patient to be treated has been diagnosed with the disease said patient is being treated for.

In an embodiment, the compound of the invention is administered in an amount of about 0.001 to about 100 mg/kg body weight per day.

A typical oral dosage is in the range of from about 0.001 to about 100 mg/kg body weight per day, preferably from about 0.01 to about 50 mg/kg body weight per day, administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

A typical oral dosage for adults is in the range of 1-100 mg/day of a compound of the present invention, such as 1-30 mg/day, or 5-25 mg/day. This may typically be achieved by the administration of 0.1-50 mg, such as 1-25 mg, such as 1, 5, 10, 15, 20, 25, 30, 40, 50 or 60 mg of the compound of the present invention once or twice daily.

A “therapeutically effective amount” of a compound as used herein means an amount sufficient to cure, alleviate or partially arrest the clinical manifestations of a given disease and its complications in a therapeutic intervention comprising the administration of said compound. An amount adequate to accomplish this is defined as “therapeutically effective amount”. The term also includes amounts sufficient to cure, alleviate or partially arrest the clinical manifestations of a given disease and its complications in a treatment comprising the administration of said compound. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. Nonetheless, prophylactic (preventive) and therapeutic (curative) treatment are two separate aspect of the invention. The patient to be treated is preferably a mammal, in particular a human being.

In one embodiment, the invention relates to the use of compound I in the manufacture of a medicament for the treatment of a disease selected from psychomotor retardation; severe depression; dysthymic disorder; cyclothymia; mood disorder due to a generalised medical condition; substance induced depression; recurrent depression; single episode depression; paediatric depression; atypical depression; post-stroke depression; exhaustion depression; depression associated with gastrointestinal pain, IBS, abuse, hostility, irritability, fatigue, anxiety (anxious depression), Lewy Body disease, Huntington's disease, or multiple sclerosis; general anxiety disorder associated with pain; seasonal affective disorder (SAD); depression or anxiety in patients with increased risk of hypertension; depression or anxiety in patients with sleep problems; stress related disorder; acute stress; dementia; mild cognitive impairment (MCI); vascular dementia; leucariosis; small vessel disease; cognitive impairment associated with affective disorders, depression, generalised depression, major depressive disorder, anxiety disorders, general anxiety disorder, panic disorder, obsessive compulsive disorder, schizophrenia, Parkinson's disease, dementia, AIDS dementia, ADHD, age associated memory impairment, Down's syndrome, epilepsy, traumatic brain injury, Asperger's syndrome, and tryptophane hydrolase gene mutations; pre-, peri-, or post menopausal dysphoric disorder; pathological crying; autism; obesity; anorexia; bulimia; binge eating; impulse control disorder; intermittent explosive disorder; kleptomania; pyromania; pathological gambling; trichotillomania; conduct disorder; burn-out; stress; chronic fatigue syndrome; circadian rhythm disorder; sleep disorder; sleep-disordered breathing; hypopnea syndrome; behavioural disturbances; behavioural disturbances in the elderly; behavioural disturbances associated with dementia; compulsive and attention spectrum disorder associated with ADHD, Asperger's syndrome and autism; aggression and agitation in dementia and Alzheimer's disease; insulin resistance associated with HPA-axis hyperactivity; whiplash; fear of flying, elevators or small rooms; and amblyopia.

In one embodiment, the invention relates to compound I for use in the treatment of a disease selected from psychomotor retardation; severe depression; dysthymic disorder;

cyclothymia; mood disorder due to a generalised medical condition; substance induced depression; recurrent depression; single episode depression; paediatric depression; atypical depression; post-stroke depression; exhaustion depression; depression associated with gastrointestinal pain, IBS, abuse, hostility, irritability, fatigue, anxiety (anxious depression), Lewy Body disease, Huntington's disease, or multiple sclerosis; general anxiety disorder associated with pain; seasonal affective disorder (SAD); depression or anxiety in patients with increased risk of hypertension; depression or anxiety in patients with sleep problems; stress related disorder; acute stress; dementia; mild cognitive impairment (MCI); vascular dementia; leucariosis; small vessel disease; cognitive impairment associated with affective disorders, depression, generalised depression, major depressive disorder, anxiety disorders, general anxiety disorder, panic disorder, obsessive compulsive disorder, schizophrenia, Parkinson's disease, dementia, AIDS dementia, ADHD, age associated memory impairment, Down's syndrome, epilepsy, traumatic brain injury, Asperger's syndrome, and tryptophane hydrolase gene mutations; pre-, peri-, or post menopausal dysphoric disorder; pathological crying; autism; obesity; anorexia; bulimia; binge eating; impulse control disorder; intermittent explosive disorder; kleptomania; pyromania; pathological gambling; trichotillomania; conduct disorder; burn-out; stress; chronic fatigue syndrome; circadian rhythm disorder; sleep disorder; sleep-disordered breathing; hypopnea syndrome; behavioural disturbances; behavioural disturbances in the elderly; behavioural disturbances associated with dementia; compulsive and attention spectrum disorder associated with ADHD, Asperger's syndrome and autism; aggression and agitation in dementia and Alzheimer's disease; insulin resistance associated with HPA-axis hyperactivity; whiplash; fear of flying, elevators or small rooms; and amblyopia.

The compounds of the present invention may be administered alone as a pure compound or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19 Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings.

Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use.

Other suitable administration forms include suppositories, sprays, ointments, cremes, gels, inhalants, dermal patches, implants, etc.

Conveniently, the compounds of the invention are administered in a unit dosage form containing said compounds in an amount of about 0.1 to 60 mg, such as 1 mg, 5 mg 10 mg, 15 mg, 20 mg or 25 mg or 30 mg or 40 mg or 50 mg of a compound of the present invention.

For parenteral routes such as intravenous, intrathecal, intramuscular and similar administration, typically doses are in the order of about half the dose employed for oral administration.

For parenteral administration, solutions of the compound of the invention in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospho lipids, fatty acids, fatty acid amines, polyoxyethylene and water. The pharmaceutical compositions formed by combining the compound of the invention and the pharmaceutical acceptable carriers are then readily administered in a variety of dosage forms suitable for the disclosed routes of administration.

Formulations of the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tablet, e.g. placed in a hard gelatine capsule in powder or pellet form or in the form of a troche or lozenge. The amount of solid carrier may vary but will usually be from about 25 mg to about 1 g.

If a liquid carrier is used, the preparation may be in the form of a syrup, emulsion, soft gelatine capsule or sterile injectable liquid such as an aqueous or non-aqueous liquid suspension or solution.

Tablets may be prepared by mixing the active ingredient with ordinary adjuvants and/or diluents followed by the compression of the mixture in a conventional tabletting machine. Examples of adjuvants or diluents comprise: Corn starch, potato starch, talcum, magnesium stearate, gelatine, lactose, gums, and the like. Any other adjuvants or additives usually used for such purposes such as colourings, flavourings, preservatives etc. may be used provided that they are compatible with the active ingredients.

Capsules comprising a compound of the present invention may be prepared by mixing a powder comprising said compound with microcrystalline cellulose and magnesium stearate and place said powder in a hard gelatine capsule. Optionally, said capsule may be coloured by means of a suitable pigment. Typically, capsules will comprise 0.25-20% of a compound of the present invention, such as 0.5-1.0%, 3.0-4.0%, or 14.0-16.0% of a compound of the present invention. These strengths can be used to conveniently deliver 1, 5, 10, 15, 20, 25, 30, 40, 50 or 60 mg of a compound of the present invention in a unit dosage form.

Solutions for injections may be prepared by dissolving the active ingredient and possible additives in a part of the solvent for injection, preferably sterile water, adjusting the solution to the desired volume, sterilising the solution and filling it in suitable ampoules or vials. Any suitable additive conventionally used in the art may be added, such as tonicity agents, preservatives, antioxidants, etc.

Compound I may be formulated in a tablet with different strengths with excipients as shown below (percentages are w/w %)

| | |
|---|---|
| Compound I, e.g. HBr salt | 1-7% |
| Calcium hydrogen phosphate, anhydrous | 35-45% |
| Maize starch | 18-23% |
| Hydroxypropylcellulose | 1-4% |
| Cellulose, microcrystalline | 25-30% |
| Sodium starch glycolate | 1-5% |
| Talc | 1-3% |
| Magnesium stearate | 0.5-2% |

Particular examples of tablets comprising compound I are

| | Strength | | |
|---|---|---|---|
| | 1 mg | 5 mg | 20 mg |
| | Core mass | | |
| | 125 mg | 125 mg | 500 mg |
| Compound I, HBr | 1.03% | 5.14% | 5.14% |
| Calcium hydrogen phophate, anhydrous | 41.21% | 39.50% | 39.5% |
| Maize starch | 21.75% | 20.35% | 20.35% |
| Hydroxypropylcellulose | 2.01% | 2.01% | 2.01% |
| Cellulose, microcrystalline | 27.50% | 27.50% | 27.50% |
| Sodium starch glycolate | 3.00% | 3.00% | 3.00% |
| Talc | 1.50% | 1.50% | 1.50% |
| Magnesium stearate | 1.00% | 1.00% | 1.00% |

Alternatively, compound I may be formulated in a tablet with different strengths with excipients as shown below (percentages are w/w %)

| | |
|---|---|
| Compound I, e.g. HBr | 1-10% |
| Mannitol | 35-50% |
| Maize starch | 18-25% |
| Hydroxypropylcellulose | 1-4% |
| Cellulose, microcrystalline | 20-25% |
| Sodium starch glycolate | 1-5% |
| Magnesium stearate | 1-5% |

Particular examples of tablets comprising compound I are

| | Strength | | |
|---|---|---|---|
| | 1 mg | 5 mg | 20 mg |
| | Core mass | | |
| | 90 mg | 90 mg | 360 mg |
| Compound I, HBr | 1.43% | 7.14% | 7.14% |
| Mannitol | 44.51% | 40.74% | 40.74% |
| Maize starch | 22.93% | 20.99% | 20.99% |
| Hydroxypropylcellulose | 2.13% | 2.13% | 2.13% |
| Cellulose, microcrystalline | 23.00% | 23.00% | 23.00% |
| Sodium starch glycolate | 3.00% | 3.00% | 3.00% |
| Magnesium stearate | 3.00% | 3.00% | 3.00% |

The tablets exemplified above may be coated, e.g. to achieve a particular colour or to make the tablets easier to swallow.

Compound I may either be administered alone or in combination with another therapeutically active compound, wherein the two compounds may either be administered simultaneously or sequentially. Examples of therapeutically active compounds which may advantageously be combined with compound I include sedatives or hypnotics, such as benzodiazepines; anticonvulsants, such as lamotrigine, valproic acid, topiramate, gabapentin, carbamazepine; mood stabilizers such as lithium; dopaminergic drugs, such as dopamine agonists and L-Dopa; drugs to treat ADHD, such as atomoxetine;

psychostimulants, such as modafinil, ketamine, methylphenidate and amphetamine; other antidepressants, such as mirtazapine, mianserin and buproprion; hormones, such as T3, estrogen, DHEA and testosterone; atypical antipsychotics, such as olanzapine and aripiprazole; typical antipsychotics, such as haloperidol; drugs to treat Alzheimer's diseases, such as cholinesterase inhibitors and memantine, folate; S-Adenosyl-Methionine; immunmodulators, such as interferons; opiates, such as buprenorphins; angiotensin II receptor 1 antagonists (AT 1 antagonists); ACE inhibitors; statins; and alphal adrenergic antagonist, such as prazosin.

Compound I may be prepared as outlined in WO 2003/029232 or in WO 2007/144006. Salts of compound I may by addition of an appropriate acid followed by precipitation. Precipitation may be brought about by e.g. cooling, removal of solvent, addition of another solvent or a mixture thereof. Alternatively, compound I may be manufactured as shown in the examples.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or a particular described aspect, unless otherwise indicated.

Unless otherwise indicated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

### EXAMPLES

Analytical Methods

X-Ray powder diffractograms (XRPD) were measured on a PANalytical X'Pert PRO X-Ray Diffractometer using CuK_{α1} radiation. The samples were measured in reflection mode in the 2θ-range 5-40° using an X'celerator detector.

Elemental composition (CHN) was measured on an Elementar Vario EL instrument from Elementar. About 4 mg of sample was used for each measurement, and the results are given as mean values of two measurements.

### Example 1A

### Serotonin (5-HT) and Norepinephrine (NE) Reuptake Inhibition

Aliquots of test compound and rat cortical synaptosome preparation were pre-incubated for 10 min/37° C., and then added [³H]NE or [³H]5-HT (final concentration 10 nM). Non-specific uptake was determined in the presence of 10 μM talsupram or citalopram and the total uptake was determined in the presence of buffer. Aliquots were incubated for 15 minutes at 37° C. After the incubation [³H]NE or [³H]5-HT taken up by synaptosomes was separated by filtration through Unifilter GF/C, presoaked in 0.1% PEI for 30 minutes, using a Tomtec Cell Harvester program. Filters were washed and counted in a Wallac MicroBeta counter.

At NET compounds of the present invention display an IC₅₀ value of 23 nM. At SERT compounds of the present invention display an IC₅₀ value of 8 nM.

### Example 1B

### 5-HT_{3A} Receptor Antagonism

In oocytes expressing human-homomeric 5-HT_{3A} receptors 5-HT activates currents with an EC₅₀ of 2600 nM. This current can be antagonised with classical 5-HT₃ antagonists such as ondansetron. Ondansetron displays a Ki value below 1 nM in this system. Compounds of the present invention exhibit potent antagonism in low concentrations (0.1 nM-100 nM) (IC₅₀˜10 nM/Kb˜2 nM) and agonistic properties when applied in higher concentrations (100-100000 nM) (EC₅₀˜2600 nM) reaching a maximal current of approximately 70-80% of the maximal current elicited by 5-HT itself. In oocytes expressing rat-homomeric 5-HT_{3A} receptors 5-HT activates currents with an EC₅₀ of 3.3 μM. The experiments were carried out as follows. Oocytes were surgically removed from mature female Xenepus laevis anaesthetized in 0.4% MS-222 for 10-15 min. The oocytes were then digested at room temperature for 2-3 hours with 0.5 mg/ml collagenase (type IA Sigma-Aldrich) in OR2 buffer (82.5 mN NaCl, 2.0 mM KCl, 1.0 mM MgCl2 and 5.0 mM HEPES, pH 7.6). Oocytes avoid of the follicle layer were selected and incubated for 24 hours in Modified Barth's Saline buffer [88 mM NaCl, 1 mM KCl, 15 mM HEPES, 2.4 mM NaHCO₃, 0.41 mM CaCl₂, 0.82 mM MgSO₄, 0.3 mM Ca(NO₃)₂] supplemented with 2 mM sodium pyruvate, 0.1 U/I penicillin and 0.1 μg/l streptomycin. Stage IV-IV oocytes were identified and injected with 12-48 nl of nuclease free water containing 14-50 pg of cRNA coding for human 5-HT3A receptors and incubated at 18° C. until they were used for electrophysiological recordings (1-7 days after injection). Oocytes with expression of human 5-HT3 receptors were placed in a 1 ml bath and perfused with Ringer buffer (115 mM NaCl, 2.5 mM KCl, 10 mM HEPES, 1.8 mM CaCl₂, 0.1 mM MgCl₂, pH 7.5). Cells were impaled with agar plugged 0.5-1 MΩ electrodes containing 3 M KCl and voltage clamped at −90 mV by a GeneClamp 500B amplifier. The oocytes were continuously perfused with Ringer buffer and the drugs were applied in the perfusate. 5-HT agonist-solutions were applied for 10-30 sec. The potencies of 5-HT₃ receptor antagonists were examined by measuring concentration-response against 10 μM 5-HT stimulation.

### Example 2A

### Compound I, HBr Salt

### 2-(4-tolylsulfanyl)-phenyl bromide

In a stirred nitrogen covered reactor N-methyl-pyrrolidone, NMP (4.5 L) was flushed with nitrogen for 20 minutes. 4-Methylbenzenethiol (900 g, 7.25 mol) was added and then 1,2-dibromobenzene (1709 g, 7.25 mol). Potassium tert-butoxide (813 g, 7.25 mol) was finally added as the last reactant. The reaction was exothermic giving a temperature rise of the reaction mixture to 70° C. The reaction mixture was then heated to 120° C. for 2-3 hours. The reaction mixture was cooled to room temperature. Ethyl acetate (4 L) was added and aqueous sodium chloride solution (15%, 2.5 L). The mixture was stirred for 20 minutes. The aqueous phase was separated and extracted with another portion of ethyl acetate (2 L). The aqueous phase was separated and the organic phases were combined and washed with sodium chloride solution (15%, 2.5 L) The organic phase was separated, dried with sodium sulphate and evaporated at reduced pressure to a red oil which contains 20-30% NMP. The oil was diluted to twice the volume with methanol and the mixture was refluxed. More methanol was added until a clear red solution was obtained. The solution was cooled slowly to room temperature while seeded. The product crystallises as off white crystals, they were isolated by filtration and washed with methanol and dried at 40° C. in a vacuum oven until constant weight.

### Ethyl 4-hydroxy-4-(2-(4-tolylsulfanyl)phenyl)-piperidin-1-carboxylate

In a stirred reactor under nitrogen cover 2-(4-tolylsulfanyl)-phenyl bromide (600 g, 2.15 mol) was suspended in heptane (4.5 L). At room temperature 10M BuLi in hexane (235 mL, 2.36 mol) was added over 10 minutes. Only a small exotherm was noticed. The suspension was stirred for 1 hour at ambient temperature and then cooled down to −40° C. 1-Carbethoxy-4-piperidone (368 g, 2.15 mol) dissolved in THF (1.5 L) was added at a rate not faster than the reaction temperature was kept below −40° C. When the reaction has gone to completion, it was warmed to 0° C. and 1M HCl (1 L) was added keeping the temperature below 10° C. The acid aqueous phase was separated and extracted with ethyl acetate (1 L). The organic phases were combined and extracted with sodium chloride solution (15%, 1 L). The organic phase was dried over sodium sulphate and evaporated to a semi crystalline mass. It was slurried with ethyl ether (250 mL) and filtered off. Dried in an vacuum oven at 40° C. until constant weight.

### Ethyl 4-(2-(4-tolylsulfanyl)phenyl)-piperidin-1-carboxylate

Trifluoroacetic acid (2.8 kg, 24.9 mol) and triethylsilane (362 g, 3.1 mol) was charged in a reactor with an efficient stirrer. Ethyl 4-hydroxy-4-(2-(4-tolylsulfanyl)phenyl)-piperidin-1-carboxylate (462 g, 1.24 mol) was added via a powder funnel in portions. The reaction was slightly exothermic. The temperature rose to 50° C. After the addition was finalised the reaction mixture was warmed to 60° C. for 18 hours. The reaction mixture was cooled down to room temperature. Toluene (750 mL) and water (750 mL) was added. The organic phase was isolated and the aqueous phase was extracted with another portion of toluene (750 mL). The organic phases were combined and washed with sodium chloride solution (15%, 500 mL) and dried over sodium sulphate. The sodium sulphate was filtered off, the filtrate evaporated at reduced pressure to a red oil which was processed further in the next step.

### 4-(2-(4-tolylsulfanyl)phenyl)-piperidin hydrobromide

The crude ethyl 4-(2-(4-tolylsulfanyl)phenyl)-piperidin-1-carboxylate as a red oil from example 3 was mixed in a stirred reactor with hydrobromic acid in acetic acid (40%, 545 mL, 3.11 mol). The mixture was heated at 80° C. for 18 hours. The reaction mixture was cooled down to room temperature. During the cooling the product crystallises out. After 1 hour at room temperature ethyl ether (800 mL) was added to the reaction mixture, and the mixture was stirred for another hour. The product was filtered off, washed with ethyl ether and dried in a vacuum oven at 50° C. until constant weight.

### Example 2B

### Compound I, HBr Salt

To 442 grams of stirred and slightly heated (approx. 45° C.) 4-(2-p-Tolylsulfanyl-phenyl)-piperidine-1-carboxylic acid ethyl ester as an oil was added 545 ml of 33 wt-% HBr in AcOH (5.7 M, 2.5 eqv.). This mixing gives a 10° C. exotherm. After final addition the reaction mixture is heated to 80° C. and left for 18 hours. A sample is withdrawn and analysed by HPLC and if not completed more 33 wt-% HBr in AcOH must be added. Otherwise the mixture is cooled to 25° C. making the product 4-(2-p-Tolylsulfanyl-phenyl)-piperidine hydrobromide to precipitate. After one hour at 25° C. the thick suspension is added 800 ml diethylether. Stirring is continued for another hour before the product is isolated by filtration, washed with 400 ml diethylether and dried in vacuum at 40° C. overnight. The hydrobromide of compound I was isolated as white solid.

### Example 2C

### Recrystallisation of Compound I, HBr Salt

A mixture of 10.0 grams of the HBr salt of compound I, e.g. prepared as above, was heated to reflux in 100 ml H₂O. The mixture became clear and fully dissolved at 80-90° C. To the clear solution was added 1 gram of charcoal and reflux was continued for 15 minutes before filtered and left to cool spontaneously to room temperature. During cooling precipitation of white solid took place and the suspension was stirred for 1 hour at room temperature. Filtration and drying in vacuum at 40° C. overnight produced 6.9 grams (69%) of the HBr acid addition salt of compound I. See FIG. 1 for XRPD. Elemental analysis: 3.92% N, 59.36% C, 6.16% H (theory: 3.85% N, 59.34% C, 6.09% H)

### Example 3

### Compound I, Further Salts

Preparation of Stock-Solutions of Free Base

A mixture of 500 ml ethyl acetate and 200 ml H₂O was added 50 grams of the HBr salt of compound I producing a two-phased slurry. To this slurry was added approximately 25 ml conc. NaOH that caused formation of a clear two-phased solution (pH was measured to 13-14). The solution was stirred vigorously for 15 minutes and the organic phase was separated. The organic phase was washed with 200 ml H₂O, dried over Na₂SO₄, filtered and evaporated in vacuum at 60° C. acieving the free base in 38 grams yield (99%) as an almost colourless oil.

Dissolving 10 grams of the oil and adjusting the volume to 150 ml using ethyl acetate produced a 0.235 M stock-solution in ethyl acetate from which aliquots of 1.5 ml (100 mg of the free base) was used.

Dissolving 10 grams of the oil and adjusting the volume to 100 ml using 96-vol % EtOH produced a 0.353 M stock-solution in EtOH from which aliquots of 1.0 ml (100 mg of the free base) was used.

Formation of Salts Using Stock-Solutions of the Free Base

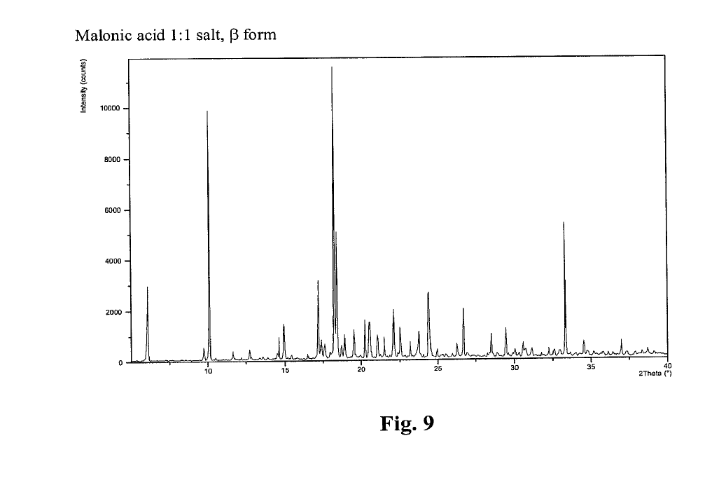

The given aliquots were placed in test tubes and while stirred the appropriate amount of acid was added as indicated in Table 1. If the acid is a liquid it was added neat otherwise it was dissolved in the given solvent prior to addition. After mixing and precipitation stirring was continued overnight and the precipitate collected by filtration. Before drying in vacuum at 30° C. a small reference sample was withdrawn and dried at room temperature without vacuum. This procedure was included in order to test for solvates. Some results are presented in Table 1. Selected XRPD diffractograms are shown in FIGS. 1-9, and major peak positions are tabulated in Table 2. Table 3 shows the solubility of compounds used in the present invention in water together with pH in the resulting saturated solution. The column “Precipitate” shows whether the precipitate isolated after the solubility determination is identical to the compound dissolved, which is indicative of the formation of hydrates.

TABLE 1

| | | Amount | | | | | | | |
|---|---|---|---|---|---|---|---|---|---|
| | MW | of Acid | | | | | | | |
| Acid (Base:Acid) | (g/mol) | (mg or μl) | Solvent | CHN (exp.) | | | CHN (theory) | | |
| Palmitic acid, hexadecanoic | 256.42 | 90.5 | EtOAc | 75.36 | 9.77 | 2.46 | 75.64 | 9.9 | 2.6 |
| acid 1:1 | | | | | | | | | |
| DL-Lactic acid, DL-2- | 90.1 | 31.8 | EtOAc | 66.88 | 7.26 | 3.52 | 67.53 | 7.29 | 3.75 |
| hydroxypropionic acid 1:1 | | | | | | | | | |
| Adipicacid, 1,6-hexanedioic | 146.14 | 51.6 | EtOAc | 66.08 | 7.23 | 2.98 | 67.1 | 7.27 | 3.26 |
| acid 1:1 | | | | | | | | | |
| Adipicacid, 1,6-hexanedioic | 146.14 | 25.8 | EtOAc | 70.66 | 7.32 | 3.82 | 70.75 | 7.35 | 3.93 |
| acid 2:1 | | | | | | | | | |
| Fumaric acid 1:1 | 116.01 | 40.9 | EtOH | 65.71 | 6.41 | 3.35 | 66.14 | 6.31 | 3.51 |
| Glutaric acid, 1,5- | 132.12 | 46.6 | EtOAc | 66.09 | 6.97 | 3.2 | 66.48 | 7.03 | 3.37 |
| pentanedioic acid 1:1 | | | | | | | | | |
| Malonic acid 1:1 | 104.1 | 36.7 | EtOAc | 65.04 | 6.53 | 3.54 | 65.09 | 6.5 | 3.62 |
| Oxalic acid 1:1 | 90.1 | 31.8 | EtOH | 64.28 | 6.41 | 3.61 | 64.32 | 6.21 | 3.75 |
| Sebacoinic acid, 1,8- | 202.02 | 35.6 | EtOAc | 71.79 | 7.86 | 3.58 | 71.83 | 7.86 | 3.64 |
| octanedioic acid 2:1 | | | | | | | | | |
| Succinic acid, 1,4- | 118.1 | 20.8 | EtOAc | 65.65 | 6.86 | 3.4 | 65.80 | 6.78 | 3.49 |
| butanedioic acid, 2:1 | | | | | | | (1:1 salt formed) | | |
| L-malic acid, L-2-hydroxy | 134.1 | 47.3 | EtOAc | 62.87 | 6.20 | 3.22 | 63.29 | 6.52 | 3.36 |
| butanedioic acid 1:1, α | | | | | | | | | |
| L-malic acid, L-2-hydroxy | 134.1 | 47.3 | EtOH | 62.99 | 6.66 | 3.13 | 63.29 | 6.52 | 3.36 |
| butanedioic acid 1:1, β | | | | | | | | | |
| D-tartaric acid, D-2,3- | 150.1 | 53.0 | EtOH | 60.67 | 6.4 | 3.07 | 60.95 | 6.28 | 3.23 |
| dihydroxy butanedioic acid | | | | | | | | | |
| 1:1 | | | | | | | | | |
| L-aspartic acid 1:1 | 133.1 | 47.0 | EtOH | 59.31 | 6.7 | 7.1 | 63.43 | 6.78 | 6.73 |
| | | | | (contains excess of | | | | | |
| | | | | acid) | | | | | |
| Glutamic acid 1:1 | 165.15 | 58.3 | EtOH | 56.38 | 6.88 | 7.35 | 56.46 | 6.94 | 7.06 |
| | | | | (contains excess of | | | (for 1:1-salt and acid- | | |
| | | | | acid) | | | monohydrate 1:1) | | |
| Citric acid 2:1 | 192.13 | 33.9 | EtOAc | 65.93 | 6.72 | 3.44 | 66.46 | 6.64 | 3.69 |
| HCl/Et₂O 1:1 | 2M | 176.4 | EtOH | | | | | | |
| Phosphoric acid 1:1 | 14.7M | 24.0 | EtOAc | 55.79 | 6.47 | 3.43 | 56.68 | 6.34 | 3.67 |

TABLE 2
Selected X-ray peak positions (°2θ), 2:1 means 2
bases to 1 acid. All values +−0.1°

| | | | | |
|---|---|---|---|---|
| Palmitate | 7.00 | 16.34 | 22.73 | 28.21 |
| Stearate | 6.70 | 15.52 | 21.81 | 28.91 |
| DL-Lactate | 5.30 | 8.18 | 9.44 | 17.24 |
| Lactate hydrate | 11.67 | 16.70 | 18.25 | 21.76 |
| hydroxyl-isobutyrate | 5.09 | 16.60 | 20.38 | 27.37 |
| Sebacoin acid salt | 7.18 | 12.53 | 21.11 | 24.19 |
| Adipinic acid salt 2:1 | 8.03 | 13.52 | 17.90 | 24.60 |
| Adipinic acid salt 1:1 α | 9.33 | 14.01 | 18.72 | 20.63 |
| Adipinic acid salt 1:1 β | 15.69 | 21.53 | 25.81 | 31.18 |
| Glutarate 1:1 | 9.39 | 11.70 | 14.05 | 14.58 |
| Succinate 1:1 | 11.74 | 14.33 | 17.75 | 26.84 |
| Fumarate 1:1 | 8.90 | 11.47 | 19.25 | 22.33 |
| Fumarate 2:1 | 8.49 | 12.48 | 17.78 | 23.97 |
| Maleate 1:1 | 12.11 | 15.51 | 17.48 | 22.53 |
| Maleate 1:1 hydrate | 12.81 | 18.76 | 20.53 | 27.31 |
| Malonate α | 10.77 | 16.70 | 19.93 | 24.01 |
| Malonate β | 6.08 | 10.11 | 18.25 | 20.26 |
| L-Aspartate | 11.05 | 20.16 | 20.60 | 25.00 |
| L-Aspartate hydrate | 7.80 | 13.80 | 14.10 | 19.63 |
| Glutamate | 7.71 | 14.01 | 19.26 | 22.57 |
| Oxalate | 14.68 | 17.45 | 19.50 | 23.90 |
| Malate 1:1 α | 8.30 | 12.04 | 17.23 | 20.67 |
| Malate 1:1 β | 10.91 | 12.87 | 14.14 | 26.16 |
| Malate hydrate | 12.30 | 15.56 | 19.56 | 23.30 |
| D-tartrate (from EtOH) | 5.08 | 17.18 | 19.42 | 22.10 |
| Hydrochloride | 12.44 | 16.72 | 19.45 | 25.02 |
| Hydrobromide | 6.08 | 14.81 | 19.26 | 25.38 |
| Hydrobromide 1-PrOH solvate | 6.57 | 13.12 | 19.07 | 24.77 |

TABLE 3

| | Solubility | | |
|---|---|---|---|
| Acid (Base:Acid) | (mg/ml) | Resulting pH | Precipitate |
| Palmitic acid, hexadecanoic | 0.4 | 8.6 | =start |
| acid 1:1 | | | |
| DL-Lactic acid, DL-2- | >150 | 6.1 | =start (after |
| hydroxypropionic acid 1:1 | | | evaporation) |
| Adipicacid, 1,6-hexanedioic | 2.5 | 4.0 | Partly 2:1 salt |
| acid 1:1 | | | |
| Adipicacid, 1,6-hexanedioic | 1.0 | 7.8 | =start |
| acid 2:1 | | | |
| Fumaric acid 1:1 | 0.2 | 3.3 | =start |
| Glutaric acid, 1,5- | 13 | 4.6 | =start |
| pentanedioic acid 1:1 | | | |
| Malonic acid 1:1 (α) | 5.2 | 4.0 | =new form (β) |
| Oxalic acid 1:1 | 1.1 | 2.7 | =Start |
| Sebacoinic acid, 1,8- | 0.7 | 5.5 | =Start |
| octanedioic acid 2:1 | | | |
| Succinic acid, 1,4- | 2.0 | 4.0 | Hydrate |
| butanedioic acid, 2:1 | | | |
| L-malic acid, L-2-hydroxy | 2.8 | 4.0 | Hydrate |
| butanedioic acid 1:1, β | | | |
| D-tartaric acid, D-2,3- | 1.8 | 3.5 | Hydrate |
| dihydroxy butanedioic acid | | | |
| 1:1 | | | |
| L-aspartic acid 1:1 | 39 | 4.3 | Hydrate |
| Glutamic acid 1:1 | >35 | 4.6 | — |
| Citric acid 2:1 | 0.5 | 4.7 | =Start |
| Phosphoric acid 1:1 | 6.0 | 2.0 | ? |
| HCl | 4.5 | 6.8 | =Start |
| HBr | 2.4 | 7.0 | =Start |

### Example 4

### Effect on Acetylcholine Levels

The experiment was designed to evaluate the effects of compounds of the present invention on extracellular levels of acetylcholine in the prefrontal cortex and ventral hippocampus of freely-moving rats.

Male Sprague-Dawley rats, initially weighing 275-300 g, were used. The animals were housed under a 12-hr light/dark cycle under controlled conditions for regular in-door temperature (21±2° C.) and humidity (55±5%) with food and tap water available ad libitum.

Surgery and Microdialysis Experiments

Rats were anaesthetised with hypnorm/dormicum (2 ml/kg) and intracerebral guide cannulas (CMA/12) were stereotaxically implanted into the hippocampus, aiming to position the dialysis probe tip in the ventral hippocampus (co-ordinates: 5.6 mm posterior to bregma, lateral −5.0 mm, 7.0 mm ventral to dura or in the frontal cortex (co-ordinates: 3.2 mm anterior to bregma; lateral, 0.8 mm; 4.0 mm ventral to dura). Anchor screws and acrylic cement were used for fixation of the guide cannulas. The body temperature of the animals was monitored by rectal probe and maintained at 37° C. The rats were allowed to recover from surgery for 2 days, housed singly in cages. On the day of the experiment a microdialysis probe (CMA/12, 0.5 mm diameter, 3 mm length) was inserted through the guide cannula.

The probes were connected via a dual channel swivel to a microinjection pump. Perfusion of the microdialysis probe with filtered Ringer solution (145 mm NaCl, 3 mM KCl, 1 mM MgCl₂, 1.2 mM CaCl₂ containing 0.5 μM neostigmine) was begun shortly before insertion of the probe into the brain and continued for the duration of the experiment at a constant flow rate of 1 μl/min. After 180 min of stabilisation, the experiments were initiated. Dialysates were collected every 20 min. After the experiments the animals were sacrificed, their brains removed, frozen and sliced for probe placement verification.

Analysis of Dialysate Acetylcholine

Concentration of acetylcholine (ACh) in the dialysates was analysed by means of HPLC with electrochemical detection using a mobile phase consisting of 100 mM disodium hydrogenphosphate, 2.0 mM octane sulfonic acid, 0.5 mM tetramethyl-ammonium chloride and 0.005% MB (ESA), pH 8.0. A pre-column enzyme reactor (ESA) containing immobilised choline oxidase eliminated choline from the injected sample (10 μl) prior to separation of ACh on the analytical column (ESA ACH-250); flow rate 0.35 ml/min, temperature: 35° C. After the analytical column the sample passed through a post-column solid phase reactor (ESA) containing immobilised acetylcholineesterase and choline oxidase. The latter reactor converted ACh to choline and subsequently choline to betaine and H₂O₂. The latter was detected electrochemical by using a platinum electrode (Analytical cell: ESA, model 5040).

Data Presentation

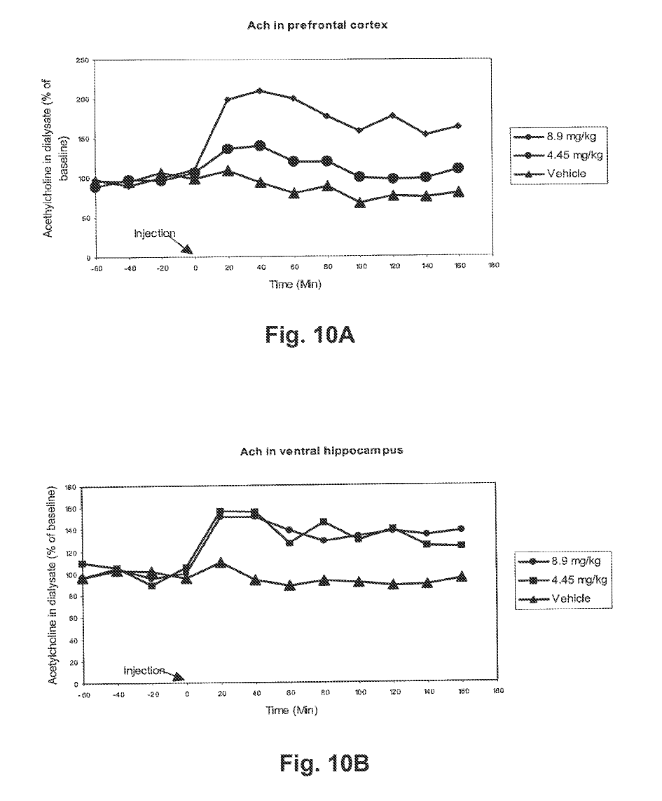

In single injection experiments the mean value of 3 consecutive ACh samples immediately preceding compound administration served as the basal level for each experiment and data were converted to percentage of basal (mean basal pre-injection values normalized to 100%). The data are presented in FIGS. 10A and 10B.

The data presented in FIGS. 10A and 10B shows a dose dependent increase in the extra-cellular acetylcholine levels in the brain. This pre-clinical finding is expected to translate into an improvement in cognition in a clinical setting useful e.g. in the treatment of cognitive impairment and diseases characterised by a cognitive impairment.

### Example 5

### Effect on Dopamine Levels

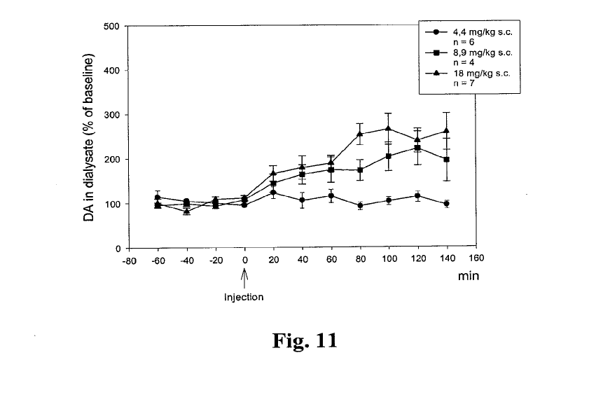

A single injection of compounds of the present invention dose-dependently increased extracellular dopamine (DA) levels in the rat frontal cortex. The compound of the present invention at 8.9 mg/kg and 18 mg/kg s.c., enhanced the DA levels by approximately 100% and 150%, respectively, above baseline levels as depicted in FIG. 11. Amounts are calculated as the free base.

Method.

Male Sprague-Dawley rats, initially weighing 275-300 g, were used. The animals were housed under a 12-hr light/dark cycle under controlled conditions for regular in-door temperature (21±2° C.) and humidity (55±5%) with food and tap water available ad libitum. For the three-day treatment experiments osmotic minipumps (Alzet, 2ML1) were used. The pumps were filled under aseptic conditions and implanted subcutaneously under sevoflurance anaesthesia. The experiments were carried out with the minipumps on board. Blood samples for measuring plasma levels of the test compound after 3 days of treatment were collected at the end of the experiments.

Surgery and Microdialysis Experiments.

Animals were anaesthetised with hypnorm/dormicum (2 ml/kg) and intracerebral guide cannulas (CMA/12) were stereotaxically implanted into the hippocampus, positioning the dialysis probe tip in the ventral hippocampus (co-ordinates: 5.6 mm anterior to bregma, lateral −5.0 mm, 7.0 mm ventral to dura or in the frontal cortex (co-ordinates: 3.2 mm anterior to bregma; lateral, 3.0 mm; 4,0 mm ventral to dura). Anchor screws and acrylic cement were sued for fixation of the guide cannulas. The body temperature of the animals was monitored by rectal probe and maintained at 37° C. The rats were allowed to recover from surgery for 2 days, housed singly in cages. On the day of the experiment a microdialysis probe (CMA/12, 0.5 mm diameter, 3 mm length) was inserted through the guide cannula. The probes were connected via a dual channel swivel to a microinjection pump. Perfusion of the microdialysis probe with filtered Ringer solution (145 mm NaCl, 3 mM KCl, 1 mM MgCl₂, 1.2 mM CaCl₂) was begun shortly before insertion of the probe into the brain and continued for the duration of the experiment at a constant flow rate of 1 (1.3) μL/min. After 180 min of stabilisation, the experiments were initiated. Dialysates were collected every 20 (30) min. After the experiments the rats were sacrificed by decapitation, their brains removed, frozen and sliced for probe placement verification.

Analysis of Dialysates.

Concentration of dopamine in the dialysates was analysed by means of HPLC with electrochemical detection. The monoamines were separated by reverse phase liquid chromatography (ODS 150×3 mm, 3 μM). Dopamine: Mobile phase consisting of 90 mM NaH₂PO₄, 50 mM sodium citrate, 367 mg/l sodium 1-octanesulfonic acid, 50 μM EDTA and 8% acetonitrile (pH 4.0) at a flow rate of 0.5 ml/min. Electrochemical detection was accomplished using a coulometric detector; potential set at 250 mV (guard cell at 350 mV) (Coulochem II, ESA).

### Example 6

### Effect on Neuropatic Pain

To demonstrate an efficacy against neuropathic pain, the compound of the present invention was tested in the formalin model of neuropathic pain [Neuropharm., 48, 252-263, 2005; Pain, 51, 5-17, 1992]. In this model, mice receive an injection of formalin (4.5%, 20 μl) into the plantar surface of the left hind paw and afterwards are placed into individual glass beakers (2 l capacity) for observation. The irritation caused by the formalin injection elicits a characteristic biphasic behavioural response, as quantified by the amount of time spent licking the injured paw. The first phase (˜0-10 minutes) represents direct chemical irritation and nociception, whereas the second (˜20-30 minutes) is thought to represent pain of neuropathic origin. The two phases are separated by a quiescent period in which behaviour returns to normal. Measuring the amount of time spent licking the injured paw in the two phases assesses the effectiveness of test compounds to reduce the painful stimuli.

Eight C57/B6 mice (ca. 25 g) were tested per group. Table 4 below show the amount of time spent licking the injured paw in the two phases, i.e. 0-5 minutes and 20-30 minutes post formalin injection. The amount of compound administered is calculated as the free base.

TABLE 4

| | Vehicle | 1.0 mg/kg | 2.5 mg/kg | 10 mg/kg |
|---|---|---|---|---|
| 0-5 minutes (sec) | 42 | 37 | 30 | 37 |
| 20-30 minutes (sec) | 41 | 43 | 26 | 6 |

The data in table 4 shows that the compound of the present invention has little effect in the first phase representing direct chemical irritation and nociception. More notably, the data also show a clear and dose dependent decrease in the time spent licking paws in the second phase indicating an effect of the compound of the present invention in the treatment of neuropathic pain.

1. A method for treating a disease selected from psychomotor retardation; severe depression; dysthymic disorder; cyclothymia; mood disorder due to a generalised medical condition; substance induced depression; recurrent depression; single episode depression; paediatric depression; atypical depression; post-stroke depression; exhaustion depression; depression associated with gastrointestinal pain, IBS, abuse, hostility, irritability, fatigue, anxiety (anxious depression), Lewy Body disease, Huntington's disease, or multiple sclerosis; general anxiety disorder associated with pain; seasonal affective disorder (SAD); depression or anxiety in patients with increased risk of hypertension; depression or anxiety in patients with sleep problems; stress related disorder; acute stress; dementia; mild cognitive impairment (MCI); vascular dementia; leucariosis; small vessel disease; cognitive impairment associated with affective disorders, depression, generalised depression, major depressive disorder, anxiety disorders, general anxiety disorder, panic disorder, obsessive compulsive disorder, schizophrenia, Parkinson's disease, dementia, AIDS dementia, ADHD, age associated memory impairment, Down's syndrome, epilepsy, traumatic brain injury, Asperger's syndrome, and tryptophane hydrolase gene mutations; pre-, peri-, or post menopausal dysphoric disorder; pathological crying; autism; obesity; anorexia; bulimia; binge eating; impulse control disorder; intermittent explosive disorder; kleptomania; pyromania; pathological gambling; trichotillomania; conduct disorder; burn-out; stress; chronic fatigue syndrome; circadian rhythm disorder; sleep disorder; sleep-disordered breathing; hypopnea syndrome; behavioural disturbances; behavioural disturbances in the elderly; behavioural disturbances associated with dementia; compulsive and attention spectrum disorder associated with ADHD, Asperger's syndrome and autism; aggression and agitation in dementia and Alzheimer's disease; insulin resistance associated with HPA-axis hyperactivity; whiplash; fear of flying, elevators or small rooms; and amblyopia, the method comprising the administration of a therapeutically effective amount of 4-[2-(4-methylphenylsulfanyl)phenyl]piperidine and therapeutically acceptable salts thereof (compound I) to a patient in need thereof.

2. The method according to claim 1, wherein compound I is crystalline, provided said compound is not the hydrochloric acid salt.

3. The method according to claim 1, wherein compound I is the hydrobromic acid salt.

4. The method according to claim 3, wherein compound I is crystalline with XRPD peaks at approximately 6.08, 14.81, 19.26 and 25.30°2θ.

5. The method according to claim 4, wherein compound I has an XRPD as depicted in FIG. 1.

6. The method according to claim 1, wherein compound I is administered to said patient in a unit dose of about 1-60 mg.

7. The method according to claim 6, wherein the hydrobromic acid salt of 4-[2-(4-methylphenylsulfanyl)phenyl]piperidine is administered orally to said patient at approximately 10-40 mg.

8-21. (canceled)
